(19) United States
(12) Patent Application Publication
(10) Pub. No.: US 2003/0153585 A1
(43) Pub. Date: Aug. 14, 2003

(54) Pharmaceutical preparations containing 2-pyrrolidone as the dissolving intermediary

(76) Inventors: Sven-Alexander Schreder, Heidelb erg (DE); Claudia Wildner, Bensheim (DE); Karen Schamp, Darmstadt (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/169,396

(22) PCT Filed: Jan. 5, 2001
(86) PCT No.: PCT/EP01/00081

(30) Foreign Application Priority Data
Jan. 13, 2000 (DE) 100 01 020.2

(51) Int. Cl. A61K 31/519; A61K 31/4015

(52) U.S. Cl. 514/260.1; 514/424

(57) ABSTRACT

The invention relates to pharmaceutical preparations comprising at least one medicament active ingredient and 2-pyrrolidone as solubilizer.

The invention relates to pharmaceutical preparations comprising at least one medicament active ingredient and 2-pyrrolidone as solubilizer. Preference is given to medicament active ingredients which have low solubility in water, in particular 4-[4-(3-chloro-4-methoxybenzylamino)-benzo[4,5]thieno[2,3-d]pyrimidin-2-yllcyclohexanecarboxylic acid and pharmaceutically acceptable salts thereof, 4-[4-(3-chloro-4-hydroxybenzyl-amino)benzo[4,5]thieno[2,3-d]pyrimidin-2-yl]cyclohexanecarboxylic acid and pharmaceutically acceptable salts thereof and 5-[1-(3,4-dimethoxy-benzoyl)-1,2,3,4-tetrahyd roquinolin-6-yl]-6-methyl-3,6-dihyd ro-1,3,4-thiadiazin-2-one. The preparations according to the invention are preferably administered orally. The invention furthermore relates to the use of 2-pyrrolidone as solubilizer in pharmaceutical preparations for oral administration in human medicine.

Many chemical substances exhibit poor solubility in water. If it is intended to use these low-solubility compounds as medicament active ingredients, they must be made available to the human organism in a suitable manner so that uptake of the active ingredient can take place. The bioavailability of a medicament active ingredient often depends to a considerable extent on the pharmaceutical form of the preparation.

If the medicament active ingredient is administered to the organism intravenously in the form of an injection solution, its absolute bioavailability, i.e. the proportion of the pharmaceutical species which remains unchanged in the systemic blood, i.e. enters the general circulation, is 100%.

On oral administration of a low-solubility medicament active ingredient, this must first dissolve so that it is able to overcome the entry barriers, for example the gastrointestinal tract, the oral mucous membrane, nasal membranes or the skin, in particular the stratum corneum, and can be absorbed by the body. Theoretically the best presentation form for peroral administration is a solution since in this formulation the active ingredient does not require a dissolution step before it can be absorbed.

One way of increasing the solubility of a low-solubility medicament active ingredient and thus improving absorption is comminution of the low-solubility compound. The particle size is reduced into the nanometer range and the active ingredient is thus employed in the formulation in “nanonized” form. The reduction in the particle size causes an increase in the surface area available for dissolution. Furthermore, reagents are added which modify the surface of the particles and so prevent re-aggregation. These measures cause an increase in the dissolution rate, and the concentration gradient between the active ingredient solution, for example in the stomach, and the blood increases. Known formulations in which the particle size of the medicament active ingredient has been “nanonized” in this way and processes for nanonization are described in U.S. Pat. Nos. 5,145,684, 5,534,270, 5,585,108, 5,662,883, 5,665,331 and 5,718,919.

The dissolution rate can likewise be increased by addition of a solubilizer, for example alcohols, glycerol, propylene glycol, polyethylene glycol or sorbitol.

Another possibility is the preparation of microemulsions, in which the active ingredient is in dissolved form. Known formulations which use this tech-nique and processes for the preparation of microemulsions are described in U.S. Pat. Nos. 5,141,961, 5,376,688, 5,430,017, 5,688,761, 5,505,961, 5,707,648, 5,759,566 or 5,912,011.

The invention had the object of providing novel medicaments in the form of pharmaceutical preparations for oral administration of medicament active ingredients, in particular of 4-[4-(3-chloro-4-methoxybenzylamino)benzo-[4,5]thieno[2,3-d]pyrimidin-2-yl]cyclohexanecarboxylic acid or pharmaceutically acceptable salts thereof, 4-[4-(3-chloro-4-hydroxybenzylamino)-benzo[4,5]thieno[2,3-d]pyrimidin-2-yl]cyclohexanecarboxylic acid or pharmaceutically acceptable salts thereof or 5-[1-(3,4-dimethoxybenzoyl)-1,2,3,4-tetrahydroquinolin-6-yl]-6-methyl-3,6-dihydro-1,3,4-thiadiazin-2-one, which have very good bioavailability and at the same time enable a fast increase in the concentration of the active ingredient in the body.

5-[1-(3,4-Dimethoxybenzoyl)-1,2,3,4-tetrahydroquinolin-6-yl]-6-methyl-3,6-dihydro-1,3,4-thiadiazin-2-one is a compound having positively inotropic efficacy which is known from EP 0 294 647 (Example 5). The substance has a vasodilatory action and therefore stimulates blood circulation, and also has an antithrombotic action. It can be used, in particular, for combating myocardial disorders or cardiac insufficiency.

The solubility of this compound in water is 2 μg/ml. 5-[1-(3,4-dimethoxy-benzoyl)-1,2,3,4-tetrahydroquinolin-6-yl]-6-methyl-3,6-dihydro-1,3,4-thia-diazin-2-one is a classical example of a low-solubility compound whose solubility represents the determining step for bioavailability.

The compound 4-[4-(3-chloro-4-methoxybenzylamino)benzo[4,5]thieno-[2,3-d]pyrimidin-2-yl]cyclohexanecarboxylic acid and low-solubility pharmaceutically acceptable salts thereof, in particular the ethanolamine salt, are effective inhibitors of cGMP phosphodiesterase (PDE V inhibitors). This compound, its pharmaceutically acceptable salts and a process for its preparation are described in WO 99/55708 (Example 2 or p. 9, lines 1-34). The compounds are suitable for the treatment of cardiovascular diseases, in particular cardiac insufficiency, and for the treatment and/or therapy of impotence (erectile dysfunction).

The solubility of the ethanolamine salt is 22 ng/ml in phosphate buffer (pH 1.0), 19 ng/ml in phosphate buffer (pH 7.0) and 10 ng/ml in the gastro-intestinal tract, i.e. under physiological conditions.

The solubility of the ethanolamine salt is 1 mg/100 ml in synthetic gastric juice, 6 mg/100 ml in synthetic intestinal juice, 22 ng/ml in phosphate buffer (pH 1.0), 1.6 ng/ml in phosphate buffer (pH 6) and 11.4 μg/ml in phosphate buffer (pH 10).

Analogously to the above comments, the solution of the medicament active ingredient is rate-determining for the bioavailability.

The first metabolite of 4-[4-(3-chloro-4-methoxybenzylamino)benzo[4,5]-thieno[2,3-d]pyrimidin-2-yl]cyclohexanecarboxylic acid or of the pharmaceutically acceptable salts of 4-[4-(3-chloro-4-methoxybenzylamino)benzo-[4,5]thieno[2,3-d]pyrimidin-2-yl]cyclohexanecarboxylic acid is 4-[4-(3-chloro-4-hydroxybenzylamino)benzo[4,5]thieno[2,3-d]pyrimidin-2-yl]cyclo-hexanecarboxylic acid. 4-[4-(3-Chloro-4-hydroxybenzylamino)benzo[4,5]-thieno[2,3-d]pyrimidin-2-yl]cyclohexanecarboxylic acid and pharmaceutically acceptable salts thereof are likewise effective inhibitors of cGMP phosphodiesterase (PDE V inhibitors).

Further low-solubility medicament active ingredients which are suitable for a preparation according to the invention are medicament active ingredients whose solubility in water is less than 100 μg/ml. Examples of low-solubility medicament active ingredients which are suitable for a preparation according to the invention are triamteren, phenytoin, mebendazol, glibenclamid, naproxen, nabumetone, medroxyprogesterone acetate, estradiol, lovastatin, clotrimazol, danazol, cinnarizin, loratadin, sitosterin and probucol.

The object is achieved by the discovery of the preparation according to the invention.

The invention relates to a pharmaceutical preparation comprising at least one medicament active ingredient, in particular a low-solubility medicament active ingredient, and 2-pyrrolidone as solubilizer. The pharmaceutical preparation is preferably administered orally.

The invention relates to a pharmaceutical preparation comprising at least one medicament active ingredient, in particular a low-solubility medicament active ingredient, a lipid-like assistant and 2-pyrrolidone as solubilizer.

The invention also relates to a pharmaceutical preparation comprising at least one medicament active ingredient, in particular a low-solubility medicament active ingredient, an excipient and 2-pyrrolidone as solubilizer.

The invention relates to a pharmaceutical preparation as described above, comprising at least 4-[4-(3-chloro-4-methoxybenzylamino)benzo[4,5]thieno-[2,3-d]pyrimidin-2-yl]cyclohexanecarboxylic acid or a low-solubility pharmaceutically acceptable salt thereof, and 2-pyrrolidone as solubilizer.

Preference is given to 4-[4-(3-chloro-4-methoxybenzylamino)benzo[4,5]-thieno[2,3-d]pyrimidin-2-yl]cyclohexanecarboxylic acid ethanolamine salt.

The invention relates to a pharmaceutical preparation comprising at least 4-[4-(3-chloro-4-methoxybenzylamino)benzo[4,5]thieno[2,3-d]pyrimidin-2-yl]cyclohexanecarboxylic acid or a low-solubility pharmaceutically acceptable salt thereof, 2-pyrrolidone as solubilizer and a lipid-like assistant or an excipient material.

The invention relates to a pharmaceutical preparation comprising at least 4-[4-(3-chloro-4-hydroxybenzylamino)benzo[4,5]thieno[2,3-d]pyrimidin-2-yl]cyclohexanecarboxylic acid or a low-solubility pharmaceutically acceptable salt thereof, and 2-pyrrolidone as solubilizer.

The invention relates to a pharmaceutical preparation comprising 4-[4-(3-chloro-4-hydroxybenzylamino)benzo[4,5]thieno[2,3-d]pyrimidin-2-yl]cyclo-hexanecarboxylic acid or a low-solubility pharmaceutically acceptable salt thereof, 2-pyrrolidone as solubilizer and a lipophilic assistant or an excipient material.

The invention relates to a pharmaceutical preparation comprising at least 5-[1-(3,4-dimethoxybenzoyl)-1,2,3,4-tetrahydroquinolin-6-yl]-6-methyl-3,6-dihydro-1,3,4-thiadiazin-2-one and 2-pyrrolidone as solubilizer.

The invention relates to a pharmaceutical preparation comprising at least 5-[1-(3,4-dimethoxybenzoyl)-1,2,3,4-tetrahydroq uinolin-6-yl]-6-methyl-3,6-dihydro-1,3,4-thiadiazin-2-one, 2-pyrrolidone as solubilizer and a lipophilic assistant or an excipient material.

Surprisingly, it has been found that the preparation according to the invention enables very good bioavailability of a medicament active ingredient which has low solubility in water and in gastric juice and enables a fast increase in the concentration of the active ingredient. In particular, there is no need in the preparation according to the invention to nanonize the active ingredient or to prepare a microemulsion.

Parameters which are important for the characterization of bioavailability are listed below:

AUC denotes the area under the concentration/time curve (area under the curve), where concentration denotes the concentration of the medicament active ingredient in the plasma, which is plotted against time. The AUC value is proportional to the amount of active ingredient which has entered the systemic circulation. AUC (0-6 h) refers to the area under the concentration/time curve which is determined in the time directly after ingestion up to 6 h. In AUC (0-inf), inf denotes “to infinity”.

The absolute bioavailability (BA) is given by comparison with the AUCᵢᵥ measured after intravenous administration:

BA=AUC/AUCᵢᵥ.

The relative bioavailability (BA) is obtained by comparing two medicament preparations A and B:

BAᵣₑₗ=AUC_{preparation A}/AUC_{preparation B}.

Concentration/time curves can be further characterized by means of the maximum medicament concentration cₘₐₓ in the plasma and the associated time tₘₐₓ.

In order to determine the characteristic data as just described, both in-vitro tests and in-vivo studies are carried out.

The liberation data as in-vitro test method are measured analogously to USP XXIII Method II, USP, 1995, Vol. 23/NF18, 1792, in a paddle apparatus (50 rpm) at 37° C.

The in-vivo studies are carried out as a 5-fold cross-over study in accordance with the Standard Operating Procedures (SOPs) of the Institute of Toxicology or the Grafing Institute of Pharmacokinetics and Metabolism at Merck KGaA.

In each case, the corresponding formulations according to the invention are administered orally to five male beagle hounds in the fasted state and in the fed state. After 15 or 30 minutes, and after 1, 2, 4, 6 and 24 hours, whole blood is taken and the concentration of the medicament active ingredient in the blood plasma is determined by conventional analytical methods known to the person skilled in the art.

2-Pyrrolidone is a known solubilizer which is used, for example, in U.S. Pat. No. 4,081,528 for stabilizing antibiotic aqueous solutions and in WO 97/26895 for dissolving avermectin.

U.S. Pat. No. 4,710,497 describes pyrrolidones which, in combination with a promoter, improve the ability of active ingredients to permeate through the skin.

U.S. Pat. No. 5,811,130 describes the use of 2-pyrrolidone as co-solvent in aqueous pharmaceutical solutions for injection.

Surprisingly, the use of 2-pyrrolidone in the pharmaceutical preparations according to the invention effects very good bioavailability of the low-solubility medicament active ingredients and a fast increase in the concentration of the active ingredient, characterized by the maximum concentration cₘₐₓ. The characteristic values or liberation data of a range of formulations according to the invention, described in Examples 1 and 3, compared with a range of formulations in accordance with prior-art technologies, described in Examples 2 and 4, are disclosed in Examples 5 and 6.

Besides the property of 2-pyrrolidone as solubilizer, 2-pyrrolidone is also compatible with lipid-like assistants or other suitable excipient materials. This results, in accordance with the invention, in fixing of the low-solubility medicament active ingredients dissolved in 2-pyrrolidone in a matrix of a lipid-like assistant or in a matrix of a further suitable excipient material. In general, semisolid pastes are formed, comprising the low water-solubility medicament active ingredient dissolved in 2-pyrrolidone, which can in turn be further processed in a suitable manner to give a capsule, tablet or coated tablet.

In particular, the formulation according to the invention is introduced into capsules, for example hard gelatine capsules or soft gelatine capsules.

It is likewise possible to add further excipient materials to the formulation according to the invention in the semisolid state and, after spray solidification by spray drying, for the formulation to be introduced as granules into hard gelatine capsules or soft gelatine capsules, or to be pressed with suitable tabletting assistants to give tablets. These tablets may furthermore be provided with suitable film coatings. Suitable coating materials are, for example, hydroxyp ropylmethylcellulose, hydroxypropylcellulose, vinyl copolymers, such as polyvinylpyrrolidone or polyvinyl acetate, or acrylate-methacrylate polymers.

The pharmaceutical preparation comprising at least one low-solubility medicament active ingredient selected from the group consisting of 4-[4-(3-chloro-4-methoxybenzylamino)benzo[4,5]thieno[2,3-d]pyrimidin-2-yl]cyclo-hexanecarboxylic acid and pharmaceutically acceptable salts thereof, 4-[4-(3-chloro-4-hydroxybenzylamino)benzo[4,5]thieno[2,3-d]pyrimidin-2-yl]cyclohexanecarboxylic acid and pharmaceutically acceptable salts thereof and 5-[1-(3,4-dimethoxybenzoyl)-1,2,3,4-tetrahydroquinolin-6-yl]-6-methyl-3,6-dihydro-1,3,4-thiadiazin-2-one, 2-pyrrolidone and/or a lipid-like assistant or an excipient material, can, in the semisolid state according to the invention, also be employed in ointments or gel-like medicament forms which can be applied to mucous membranes.

The term lipid-like assistant is taken to mean, for example, glycerides of polyoxyethylene or hydroxypolyoxyethylene and fatty acids having 12, 13, 14, 15, 16, 17 or 18 carbon atoms, for example oleoyl macrogol-6 glyceride, linoleoyl macrogol-6 glyceride, lauroyl macrogol-6 glyceride, caprylo-caproyl macrogol-8 glyceride, lauroyl macrogol-32 glyceride or stearoyl macrogol-32 glyceride, glycerol polyethylene glycol oxystearate, propylene glycol monolaurate or diethylene glycol monoethyl ether. Further lipid-like substances are, for example, Miglyol® neutral oils, i.e. glycerol esters of fatty acids having 8, 9 or 10 carbon atoms, or caprilic, capric or diglyceryl succinate. A commercial product of a Miglyol® neutral oil is Miglyol® 829. Usual trade names of the lipid-like assistants described above are LABRAFIL® M 1944 CS, LABRAFIL® M 2130 CS, LABRAFIL® M 2125 CS, LABRASOL®, LAUROGLYCOL®, GELUCIRE® 44/14, GELUCIRE® 50/13 or TRANSCUTOL® P from Gattefosse.

The term glyceride 1 is taken to mean oleoyl macrogol-6 glyceride or LABRAFIL® M 1944 CS.

The term glyceride 2 is taken to mean linoleoylmacrogol-6 glyceride or LABRAFIL® M 2125 CS.

The term glyceride 3 is taken to mean lauroyl macrogol-6 glyceride or LABRAFIL® M 2130 CS.

The term glyceride 4 is taken to mean caprylocaproyl macrogol-8 glyceride or LABRASOL®.

The term glyceride 5 is taken to mean lauroyl macrogol-32 glyceride or GELUCIRE® 44/14.

The term glyceride 6 is taken to mean stearoyl macrogol-32 glyceride or GELUCIRE® 50/13.

Particularly suitable for the preparation according to the invention are glyceride 3, glyceride 5 or glyceride 6. Particular preference is given to glyceride 5.

Suitable excipient materials, besides the lipid-like excipient materials, for example glyceride 5, are likewise sorbitol or microcrystalline cellulose. Particular preference is given to glyceride 5 or sorbitol.

The invention relates to a pharmaceutical preparation as described above in which the excipient material and/or the lipophilic assistant is selected from the group consisting of glyceride 5, sorbitol and glyceride 3.

If desired, further solubilizers may be a constituent of the preparation according to the invention. Suitable solubilizers are, for example, polyethylene glycols (PEG), for example PEG 1000, PEG 400 or PEG 200, or polyethylene glycol succinates, for example D-α-tocopheryl polyethylene glycol 1000 succinate, with the trade name vitamin E-TPGS®.

Further excipients or additives may be added, such as, for example, binders, dyes, glidants, sweeteners and/or aroma substances.

Suitable glidants or lubricants are, for example, talc, metal stearates, for example magnesium stearate or calcium stearate, magnesium lauryl-sulfate, polyglycols, glycerol monostearates or distearates, paraffin, cocoa butter, macrogol or leucin. Preference is given to magnesium stearate. Preferred embodiments of the preparation according to the invention comprise from 5 to 500 mg, in particular 10, 25, 50, 100 or 500 mg, of medicament active ingredient. They particularly preferably comprise 25, 50 or 100 mg of medicament active ingredient.

The proportion of 2-pyrrolidone in the preparation according to the invention is from 1 to 30 per cent by weight, preferably from 10 to 20 per cent by weight, particularly preferably from 11 to 14 per cent by weight.

The invention likewise relates to a pharmaceutical preparation as described, characterized in that it comprises from 1 to 30 per cent by weight of 2-pyrrolidone.

The invention also relates to a process for the preparation of a pharmaceutical preparation comprising a medicament active ingredient selected from the group consisting of 4-[4-(3-chloro-4-methoxybenzylamino)benzo-[4,5]thieno[2,3-d]pyrimidin-2-yl]cyclohexanecarboxylic acid and pharmaceutically acceptable salts thereof, 4-[4-(3-chloro-4-hydroxybenzylamino)-benzo[4,5]thieno[2,3-d]pyrimidin-2-yl]cyclohexanecarboxylic acid and pharmaceutically acceptable salts thereof and 5-[1-(3,4-dimethoxybenzoyl)-1,2,3,4-tetrahydroq uinolin-6-yl]-6-methyl-3,6-dihydro-1,3,4-thiadiazin-2-one, and 2-pyrrolidone, characterized in that the low-solubility medicament active ingredient is dissolved in 2-pyrrolidone, and the solution is stirred into a melt of the further assistants. The mixture can subsequently be introduced into capsules at the mixing temperature or after cooling.

The low water-solubility medicament active ingredient is dissolved in 2-pyrrolidone at temperatures of from 10° to 40° C., preferably from 15° to 25° C., particularly preferably at room temperature.

The further assistants in the preparation according to the invention are melted at temperatures of from 40° to 100° C., preferably from 50° to 70° C., particularly preferably at 60° C., and the solution comprising the low water-solubility medicament active ingredient and 2-pyrrolidone is stirred into the melt.

The mixture according to the invention prepared in this way is introduced into the hard or soft gelatin capsules immediately after the components have been mixed in the liquid state or after a certain cooling time. Introduction in the liquid state by volumetric metering is preferred.

The examples below relate to the preparation and composition of the pharmaceutical preparation according to the invention and to the preparation and composition of comparative preparations in accordance with the prior art.

In the following examples, 4-[4-(3-chloro-4-methoxybenzylamino)benzo-[4,5]thieno[2,3-d]pyrimidin-2-yl]cyclohexanecarboxylic acid, ethanolamine salt, is abbreviated to ethanolamine salt. 2-Pyrrolidone was used in the form of the commercial product Soluphor® P.

### EXAMPLE 1

Pharmaceutical preparations comprising 4-[4-(3-chloro-4-methoxybenzyl-amino)benzo[4,5]thieno[2,3-d]pyrimidin-2-yl]cyclohexanecarboxylic acid, ethanolamine salt, as medicament active ingredient. The abbreviation PEG stands for polyethylene glycol.

1. Formulation 1:

| | |
|---|---|
| Ethanolamine salt | 50 mg |
| 2-Pyrrolidone | 100 mg |
| Glyceride 5 | 780 mg |

Preparation:

A solution of 5 g of ethanolamine salt in 11 g of 2-pyrrolidone is stirred into a melt of 78 g of glyceride 5 at 60° C. The resultant mixture is introduced in the liquid state into a hard gelatin capsule, so that a capsule contains the composition of the above-mentioned formulation as a single dose.

2. Formulation 2:

| | |
|---|---|
| Ethanolamine salt | 50 mg |
| 2-Pyrrolidone | 100 mg |
| Vitamin E TGPS ® | 310 mg |
| Glyceride 3 | 160 mg |
| PEG 1000 | 350 mg |

Preparation:

31 g of vitamin E TGPS®, 16 g of glyceride 3 and 11 g of polyethylene glycol 1000 are melted at 50-70° C. to give a homogeneous mixture. A solution of 5 g of ethanolamine salt in 11 g of 2-pyrrolidone is stirred into this melt. The mixture is introduced in the liquid state into a hard gelatin capsule, so that a capsule contains the composition of the above-mentioned formulation as a single dose.

3. Formulation 3:

| | |
|---|---|
| Ethanolamine salt | 50 mg |
| 2-Pyrrolidone | 50 mg |
| PEG 200 | 100 mg |
| Sorbitol | 200 mg |

Preparation:

A mixture of 10 g of sorbitol, 5 g of polyethylene glycol 200 and 2.5 g of 2-pyrrolidone is warmed to 40-50° C. 2.5 g of ethanolamine salt are added with stirring. The mixture is introduced in the liquid state into a hard gelatine capsule, so that a capsule contains the composition of the above-mentioned formulation as a single dose.

### EXAMPLE 2

Comparative solution comprising 4-[4-(3-chloro-4-methoxybenzylamino)-benzo[4,5]thieno[2,3-d]pyrimidin-2-yl]cyclohexanecarboxylic acid, ethanol-amine salt, as low-solubility medicament active ingredient (abbreviated to ethanolamine salt), for oral administration, theoretically the best presentation form since the active compound does not require a dissolution step, but instead can be absorbed immediately:

Formulation 4:

| | |
|---|---|
| Ethanolamine salt | 50 mg |
| Hydroxypropyl-β-CD | 500 mg, |
| | where CD denotes cyclodextrin, |
| Water, purified | 2783 mg |

Preparation:

50 mg of ethanolamine salt and 500 mg of hydroxypropyl-β-cyclodextrin are dissolved in 2783 mg of water.

Formulation 5: microemulsion

| | |
|---|---|
| Ethanolamine salt | 56.3 mg |
| Glycerol stearate | 100 mg |
| Glyceride 4 | 300 mg |
| Glyceride 1 | 300 mg |
| Diethylene glycol monoethyl ether | 72 mg |
| 1,2-Propanediol | 50 mg |
| Highly disperse silicon dioxide | 21.7 mg |

Preparation:

A mixture of 1.1 g of glycerol stearate, employed as the commercial product lmwitor® 960, 3.3 g of glyceride 4, 3.3 g of glyceride 1, 792 mg of diethylene glycol monoethyl ether and 550 mg of 1,2-propanediol is warmed to 45° C. 62 mg of ethanolamine salt are added to this solution and dissolved with stirring. The highly disperse silicon dioxide is passed through a 196 mesh screen and added to the mixture. The resultant microemulsion is introduced into hard gelatin capsules so that the individual dose corresponds to the above-mentioned formulation.

### EXAMPLE 3

Pharmaceutical preparation comprising 5-[1-(3,4-dimethoxybenzoyl)-1,2,3,4-tetrahydroquinolin-6-yl]-6-methyl-3,6-dihydro-1,3,4-thiadiazin-2-one as low-solubility medicament active ingredient (abbreviated to thiadiazin-one below).

1. Formulation 6:

| | |
|---|---|
| Thiadiazinone | 30 mg |
| 2-Pyrrolidone | 100 mg |
| Glyceride 5 | 780 mg |

Preparation:

A solution of 3 g of thiadiazinone in 11 g of 2-pyrrolidone is stirred into a melt of 78 g of glyceride 5 at 60° C. The resultant mixture is introduced in the liquid state into a hard gelatin capsule so that a capsule contains the composition of the above-mentioned formulation as a single dose.

2. Formulation 7:

| | |
|---|---|
| Thiadiazinone | 30 mg |
| 2-Pyrrolidone | 100 mg |
| Glyceride 6 | 780 mg |

Preparation:

A solution of 3 g of thiadiazinone in 11 g of 2-pyrrolidone is stirred into a melt of 78g of glyceride 6 at 60° C. The resultant mixture is introduced in the liquid state into a hard gelatine capsule so that a capsule contains the composition of the above-mentioned formulation as a single dose.

3. Formulation 8:

| | |
|---|---|
| Thiadiazinone | 30 mg |
| 2-Pyrrolidone | 100 mg |
| Vitamin E TGPS ® | 310 mg |
| Glyceride 3 | 160 mg |
| PEG 1000 | 350 mg |

Preparation:

31 g of vitamin E TGPS®, 16 g of glyceride 3 and 11 g of polyethylene glycol 1000 are melted at 50-70° C. to give a homogeneous mixture. A solution of 3 g of thiadiazinone in 11 g of 2-pyrrolidone is stirred into this melt. The mixture is introduced in the liquid state into a hard gelatine capsule so that a capsule contains the composition of the above-mentioned formulation as a single dose.

### EXAMPLE 4

Comparative preparation: mixture of thiadiazinone and lactose

Formulation 9:

| | |
|---|---|
| Thiadiazinone | 30 mg |
| Lactose | 270 mg |

Preparation:

30 mg of thiadiazinone and 270 mg of lactose are mixed and introduced into a capsule.

### EXAMPLE 5

The values obtained from the cross-over study on fed and fasted animals are listed in Tables 1 and 2:

TABLE 1
50 mg of ethanolamine salt administered to the fasted
animal in formulations 1 to 4

| | | | AUC (0-6) | |
|---|---|---|---|---|
| Formulation | Cₘₐₓ [ng/ml] | tₘₐₓ[h] | [ng/ml × h] | BA [%] |
| 1 | 838 | 1.10 | 967 | 41 |
| 2 | 656 | 1.5 | 955 | 40 |
| 3 | 924 | 0.65 | 1110 | 47 |
| 4 | 966 | 0.7 | 1180 | 50 |

TABLE 2
50 mg of ethanolamine salt administered to the fed animal in
formulations 1 to 8 according to Example 5

| | | | AUC (0-6) | |
|---|---|---|---|---|
| Formulation | Cₘₐₓ [ng/ml] | tₘₐₓ[h] | [ng/ml × h] | BA [%] |
| 1 | 157 | 1.20 | 406 | 21 |
| 2 | 213 | 0.8 | 502 | 26 |
| 3 | 107 | 1.4 | 347 | 18 |
| 4 | 114 | 0.7 | 373 | 19 |
| 5 | 138 | 1.75 | 391 | |

The absolute bioavailability BA [%] is obtained from a comparison with the AUCᵢᵥ measured after i.v. administration. 1 mg/kg of ethanolamine salt was administered to the animals i.v.

The mean body weight of the animals is 14.4 kg per animal for the calculation of the bioavailability.

### EXAMPLE 6

The following in-vitro data of thiadiazinone were liberated in various liberation media at 37° C. and 50 rpm in a paddle apparatus (USP method II). The liberation volume is always 900 ml. The target dose is 30 mg of thiadiazinone, and the actual dose is determined from 6 capsules in a batch. The liberated proportion indicated in per cent is based on the actual dose. Formulation 9 serves as comparison.

Description of the Liberation Media:

1. SGF/T

| | | |
|---|---|---|
| NaCl | 2.0 | g |
| HCl (fuming) | 14 | ml |
| Triton X 100 | 1.0 | g |
| Aqua bidist. | to 1000.0 | ml |
| pH = 1.2 | | |

2. fAssif (fasted state simulated intestinal fluid)

| | | |
|---|---|---|
| NaOH (1 N) | 13.85 | ml |
| KH₂PO₄ | 3.9 | g |
| KCl | 7.7 | g |
| Na taurocholate | 1.65 | g |
| Lecithin soln. (10% in chloroform) | 5.91 | ml |
| Aqua bidist. | to 1000.0 | ml |
| pH 6.5 | | |

3. fEssif (fed state simulated intestinal fluid)

| | | |
|---|---|---|
| NaOH (1 N) | 101.0 | ml |
| Glacial acetic acid | 8.65 | g |
| KCl | 15.2 | g |
| Na taurocholate | 8.25 | g |
| Lecithin soln. (10% in chloroform) | 29.54 | ml |
| Aqua bidist. | to 1000.0 | ml |
| pH 5.0 | | |

Triton X 100 denotes polyethylene glycol mono[p-(1,1,3,3-tetramethyl-butyl)phenyl] ether.

4. Bidistilled water (aqua bidist.)

5. Texapon solution, a solution of sodium laurylsulfate in bidistilled water (12.5 mmol/l)

TABLE 3
Liberation data of formulation 6 in the liberation media
fAssif and fEssif, as described above:

| | Time (min) | | | | |
|---|---|---|---|---|---|
| | 5 | 10 | 15 | 20 | 30 |
| fEssif | 13.46 | 36.77 | 65.74 | 86.95 | 99.71 |
| [% liberation] | | | | | |
| fAssif | 4.70 | 28.57 | 49.90 | 80.21 | 92.04 |
| [% liberation] | | | | | |

| | Time (min) | | | | |
|---|---|---|---|---|---|
| | 40 | 60 | 90 | 120 | 180 |
| fEssif | 105.09 | 105.08 | 104.36 | 103.28 | 103.46 |
| [% liberation] | | | | | |
| fAssif | 98.84 | 103.15 | 104.38 | 102.98 | 101.32 |
| [% liberation] | | | | | |
| | Time (min) | | | | |
| | 210 | 240 | 300 | 360 | 420 |
| fEssif | 102.84 | 105.08 | 104.52 | 102.70 | 103.27 |
| [% liberation] | | | | | |
| fAssif | 100.82 | 99.44 | 104.38 | 98.67 | 98.85 |
| [% liberation] | | | | | |

TABLE 4
Liberation data of formulation 7 in the liberation
media SGF/T and fEssif, as described above:

| | Time (min) | | | | |
|---|---|---|---|---|---|
| | 5 | 10 | 15 | 20 | 30 |
| SGF/T | 0.24 | 0.78 | 1.73 | 3.03 | 4.77 |
| [% liberation] | | | | | |
| fAssif | 0.13 | 1.15 | 0.76 | 1.60 | 2.59 |
| [% liberation] | | | | | |
| | Time (min) | | | | |
| | 40 | 60 | 90 | 120 | 180 |
| SGF/T | 5.23 | 10.56 | 17.37 | 27.89 | 43.40 |
| [% liberation] | | | | | |
| fAssif | 3.70 | 6.81 | 15.35 | 20.80 | 28.78 |
| [% liberation] | | | | | |
| | Time (min) | | | | |
| | 210 | 240 | 300 | 360 | 420 |
| SGF/T | 54.71 | 63.51 | 76.56 | 91.13 | 98.19 |
| [% liberation] | | | | | |
| fAssif | 33.83 | 38.75 | 45.84 | 55.43 | 69.33 |
| [% liberation] | | | | | |

TABLE 5
Liberation data of formulation 8 in the liberation
media SGF/T and fEssif, as described above:

| | Time (min) | | | | |
|---|---|---|---|---|---|
| | 5 | 10 | 15 | 20 | 30 |
| SGF/T | 7.93 | 27.75 | 31.52 | 40.31 | 47.64 |
| [% liberation] | | | | | |
| fAssif | 4.25 | 13.33 | 22.77 | 31.24 | 39.71 |
| [% liberation] | | | | | |
| | Time (min) | | | | |
| | 40 | 60 | 90 | 120 | 180 |
| SGF/T | 53.24 | 65.28 | 77.80 | 86.73 | 94.05 |
| [% liberation] | | | | | |
| fAssif | 48.09 | 63.71 | 77.75 | 86.72 | 89.66 |
| [% liberation] | | | | | |
| | Time (min) | | | | |
| | 210 | 240 | 300 | 360 | 420 |
| SGF/T | 94.00 | 97.53 | 98.88 | 98.19 | 98.91 |
| [% liberation] | | | | | |
| fAssif | 89.81 | 89.25 | 88.90 | 88.55 | 88.11 |
| [% liberation] | | | | | |

TABLE 6
Liberation data of formulation 9 in the formulation media SGF/T,
fAssif and fEssif, as described above:

| | Time (min) | | | | |
|---|---|---|---|---|---|
| | 5 | 10 | 15 | 20 | 30 |
| fEssif | | 15.33 | | 24.26 | 24.63 |
| [% liberation] | | | | | |
| fAssif | | 9.48 | | 14.56 | 15.73 |
| [% liberation] | | | | | |
| SGF/T | | 12.65 | | 17.32 | 19.51 |
| [% liberation] | | | | | |
| | Time (min) | | | | |
| | 45 | 60 | 90 | 120 | 180 |
| fEssif | 26.67 | 27.85 | 33.18 | 36.00 | 43.48 |
| [% liberation] | | | | | |
| fAssif | 17.51 | 18.49 | 19.39 | 19.79 | 19.49 |
| [% liberation] | | | | | |
| SGF/T | 20.88 | 23.68 | 22.60 | 25.29 | 25.79 |
| [% liberation] | | | | | |
| | Time (min) | | | | |
| | 210 | 240 | 300 | 360 | 420 |
| fEssif | | 43.40 | 43.39 | 43.73 | 41.66 |
| [% liberation] | | | | | |
| fAssif | | 19.46 | 19.95 | 20.09 | 20.41 |
| [% liberation] | | | | | |
| SGF/T | | 26.07 | 27.48 | 25.59 | 26.05 |
| [% liberation] | | | | | |

1. Pharmaceutical preparation comprising at least one medicament active ingredient and 2-pyrrolidone as solubilizer.

2. Pharmaceutical preparation according to claim 1, comprising at least the medicament active ingredient 4-[4-(3-chloro-4-methoxy-benzylamino)benzo[4,5]thieno[2,3-d]pyrimidin-2-yl]cyclohexane-carboxylic acid or a low-solubility pharmaceutically acceptable salt thereof.

3. Pharmaceutical preparation according to claim 1, comprising at least the medicament active ingredient 4-[4-(3-chloro-4-hydroxy-benzylamino)benzo[4,5]thieno[2,3-d]pyrimidin-2-yl]cyclohexane-carboxylic acid or a low-solubility pharmaceutically acceptable salt thereof.

4. Pharmaceutical preparation according to claim 1, comprising at least 5-[1-(3,4-dimethoxybenzoyl)-1,2,3,4-tetrahydroquinolin-6-yl]-6-methyl-3,6-dihydro-1,3,4-thiadiazin-2-one.

5. Pharmaceutical preparation according to claims 1 to 4, characterized in that an excipient material and/or a lipophilic assistant are present.

6. Pharmaceutical preparation according to claim 5, characterized in that glyceride 5, sorbitol or glyceride 3 is present.

7. Process for the preparation of a pharmaceutical preparation according to claims 1 to 6, characterized in that the medicament active ingredient is dissolved in 2-pyrrolidone, and the solution is stirred into the melt of the further assistants.

8. Use of 2-pyrrolidone as solubilizer in pharmaceutical preparations for oral administration in human medicine.
